(19) United States
(12) Patent Application Publication
(10) Pub. No.: US 2010/0036185 A1
(43) Pub. Date: Feb. 11, 2010

(54) PRODUCTION METHOD OF ETHYLENE LOW POLYMER

(75) Inventors: Kazuyuki Yokoyama, Okayama (JP); Hiroki Emoto, Okayama (JP)

Correspondence Address: OBLON, SPIVAK, MCCLELLAND MAIER & NEUSTADT, L.L.P., 1940 DUKE STREET, ALEXANDRIA, VA, 22314, US

(73) Assignee: Mitsubishi Chemical Corporation, Tokyo (JP)

(21) Appl. No.: 12/519,778

(22) PCT Filed: Oct. 25, 2007
(86) PCT No.: PCT/JP2007/070852

§ 371 Date: Jun. 18, 2009

(30) Foreign Application Priority Data
Dec. 28, 2006 (JP) 2006-356464

(51) Int. Cl. C07C 2/08

(52) U.S. Cl. 585/510

(57) ABSTRACT

The object of the present invention is to provide a method of recovering decene under the condition that a decomposition product of a chromium series catalyst and the like is difficult to form from a high boiler from which an ethylene low polymer has been separated, in a production method of an ethylene low polymer using a chromium series catalyst. The present invention relates to that an ethylene low polymer and a high boiler containing a chromium series catalyst, decene, tetradecene and a by-produced polymer are separated by evaporation operation from a reaction liquid containing an ethylene low polymer obtained by low polymerization reaction using a chromium series catalyst, and the high boiler is concentrated such that the tetradecene concentration is 5% by weight or more by an evaporative separator 70 and a liquid storage tank 80, and additionally, decene is evaporated and separated so as to satisfy the following general expression (1) wherein T is temperature (° C.) of a residual solution, and θ is residence time (min.) of a residual solution.

[Exp. 1]

θ/1.2EXP(850/T)≦1 (1)

### TECHNICAL FIELD

The present invention relates to a production method of an ethylene low polymer. More particularly, it relates to a production method of an ethylene low polymer such as 1-hexene.

### BACKGROUND ART

Conventionally, a production method in which an α-olefin low polymer such as 1-hexene is selectively obtained using an α-olefin such as ethylene as a raw material and using a chromium series catalyst is known.

For example, Patent Document 1 reports a production method in which an α-olefin low polymer mainly comprising 1-hexene is obtained in high yield and high selectivity using a chromium series catalyst comprising a chromium compound, a nitrogen-containing compound such as an amine, an alkylaluminum compound and a halogen-containing compound (see Patent Document 1 and Patent Document 2).

Patent Document 1: JP-A-08-003216

Patent Document 2: JP-A-10-109946

### DISCLOSURE OF THE INVENTION

### Problems that the Invention is to Solve

By the way, in the case of an ethylene low polymer using ethylene as a raw material, decene, tetradecene and by-produced polymers such as polyethylene are formed as side reaction products. Such side reaction products are separated from a reaction liquid together with a chromium series catalyst. At that time, decene, tetradecene, and by-produced polymers such as polyethylene separated are generally discarded together with a chromium series catalyst.

However, there is the case that decene discarded is recovered, and is effectively utilized as a product such as a fuel or a solvent, or by recycling as a solvent.

Thus, to effectively utilize decene, the kind and/or amount of impurities contained in decene recovered give rise to the problem.

In particular, in a production method of obtaining an α-olefin low polymer using a chromium series catalyst, a nitrogen compound and a halogen compound are used as constituents of the chromium series catalyst. Therefore, there is the great possibility that those compounds introduce into decene recovered. Furthermore, where concentration separation operation is conducted under high temperature condition, there is the case that decomposition products of the chromium series catalyst introduce into decene recovered.

The present invention has been made to solve the technical problem in such a production method of an ethylene low polymer.

Accordingly, an object of the present invention is to provide a method of recovering decene under the condition that it is difficult to form decomposition products of a chromium series catalyst from a residual solution from which an unreacted ethylene, 1-hexene and a solvent have been separated, in a production method of an ethylene low polymer using a chromium series catalyst.

### Means for Solving the Problems

As a result of extensive and intensive investigation to solve the above problems, the present inventors have reached to achieve the present invention. That is, the gist of the present invention resides in the following (1) to (5).

(1) A production method of an ethylene low polymer using a chromium series catalyst, characterized in that:

ethylene is subjected to low polymerization in a solvent in the presence of the chromium series catalyst,

an ethylene low polymer is separated from a reaction liquid containing the ethylene low polymer to obtain a solution containing decene and tetradecene, and

decene is separated and recovered from the solution containing decene and tetradecene by an evaporative separator under the condition of the following general expression (1):

[Exp. 1]

θ/1.2EXP(800/T)≦1 (1)

(wherein T is temperature (° C.) of a residual solution, and θ is residence time (min.) of a residual solution in an evaporative separator.)

(2) The production method of an ethylene low polymer described in (1), characterized in that the amount of a halogen contained in the decene separated from the evaporative separator is decomposition rate of 10% or less to the amount of a halogen contained in the residual solution.

(3) The production method of an ethylene low polymer described in (1) or (2), characterized in that the chromium series catalyst is constituted of a combination of at least (a) a chromium compound, (b) a nitrogen-containing compound, (c) an aluminum-containing compound and (d) a halogen-containing compound.

(4) The production method of an ethylene low polymer described in any one of (1) to (3), characterized in that the evaporative separator is a thin film evaporator.

(5) The production method of an ethylene low polymer described in any one of (1) to (4), characterized in that the ethylene low polymer is 1-hexene.

Thus, according to the present invention, there is provided a production method of an ethylene low polymer using a chromium series catalyst, characterized in that ethylene is subjected to low polymerization in a solvent in the presence of the chromium series catalyst, an ethylene low polymer is separated from a reaction liquid containing the ethylene low polymer to obtain a solution containing decene and tetradecene, and decene is separated and recovered from the solution containing decene and tetradecene by an evaporative separator under the condition of the following general expression (1):

[Exp. 2]

θ/1.2EXP(800/T)≦1 (1)

(wherein T is temperature (° C.) of a residual solution, and θ is residence time (min.) of a residual solution in an evaporative separator.)

In the production method of an ethylene low polymer to which the present invention is applied, the amount of a halogen contained in the decene separated from the evaporative separator is preferably run-down rate of 10% or less to the amount of a halogen contained in the residual solution.

In the production method of an ethylene low polymer to which the present invention is applied, the chromium series catalyst is preferably constituted of a combination of at least (a) a chromium compound, (b) a nitrogen-containing compound, (c) an aluminum-containing compound and (d) a halogen-containing compound.

Furthermore, the ethylene low polymer is preferably 1-hexene.

### ADVANTAGE OF THE INVENTION

According to the present invention, decene having a reduced content of a decomposition product of the chromium series catalyst can be recovered from a residual solution from which an unreacted ethylene, 1-hexene and a solvent have been separated (called a high boiler or a high boiling by-product liquid).

### BRIEF DESCRIPTION OF THE DRAWINGS

FIG. 1 is a view explaining a production flow example of an ethylene low polymer in the embodiment of the invention.

FIG. 2 is a view explaining a flow example of evaporative separation of a high boiler by an evaporative separator.

FIG. 3 is a view explaining a range of the general expression (1) which conducts an evaporative separation operation in the embodiment of the invention.

### DESCRIPTION OF REFERENCE NUMERALS AND SIGNS

10 . . . Reactor
10a . . . Stirring machine
11, 22, 32, 41, 42, 51, 80a . . . Piping
11a . . . Deactivator supply piping
12 . . . First supply piping
12a . . . Ethylene supply piping
13 . . . Second supply piping
13a . . . Catalyst supply piping
14 . . . Third supply piping
15 . . . Fourth supply piping
21, 31 . . . Circulation piping
16, 81 . . . Condenser
17 . . . Compressor
20 . . . Degassing tank
30 . . . Ethylene separation column
40 . . . High boiling separation column
42c . . . High boiler feed
50 . . . Hexene separation column
52 . . . Solvent circulation piping
60 . . . Solvent drum
70 . . . Evaporative separator
80 . . . Liquid storage tank
80c . . . Gear pump
A . . . Bottom
B . . . Distillate
C . . . Industrial waste

### BEST MODE FOR CARRYING OUT THE INVENTION

The best mode for carrying out the invention (hereinafter, the embodiment of the invention) is described in detail below. The invention is not limited to the following embodiment, and can be carried out with various modifications within a scope of its gist. Furthermore, the drawings used are to explain the present embodiment, and do not show the actual size.

### (Ethylene)

In the production method of an ethylene low polymer to which the embodiment of the invention is applied, when ethylene is used as a raw material, impurity components other than ethylene may be contained in the raw material. Specific impurity components include methane, ethane, acetylene and carbon dioxide. Those components are preferably in an amount of 0.1 mol % or less based on ethylene of the raw material.

### (Chromium Series Catalyst)

The chromium series catalyst is descried below. The chromium series catalyst used in the embodiment of the invention includes a catalyst constituted of a combination of at least a chromium compound (a), at least one nitrogen-containing compound (b) selected from the group consisting of an amine, an amide and an imide, and an aluminum-containing compound (c).

The chromium series catalyst used in the embodiment of the invention may contain a halogen-containing compound (d) as the fourth component according to need. Each component is described below.

### (Chromium Compound (a))

The chromium compound (a) used in the embodiment of the invention includes at least one compound represented by the general formula CrXₙ. In the general formula, X represents an optional organic group or inorganic group, or a negative atom, and n is an integer of from 1 to 6, and is preferably 2 or more. When n is 2 or more, X may be the same or different.

Examples of the organic group include a hydrocarbon group having from 1 to 30 carbon atoms, a carbonyl group, an alkoxy group, a carboxyl group, a β-diketonate group, a β-ketocarboxyl group, a β-ketoester group and an amido group.

Examples of the inorganic group include chromium salt-forming groups such as a nitric acid group or a sulfuric acid group. Examples of the negative atom include oxygen and a halogen. A halogen-containing chromium compound is not included in the halogen-containing compound (d) described hereinafter.

The number of valency of chromium (Cr) is 0 to 6. The preferred chromium compound (a) includes a carboxylate of chromium (Cr). Specific examples of the carboxylate of chromium include chromium (II) acetate, chromium (III) acetate, chromium (III)-n-octanoate, chromium (III)-2-ethylhexanoate, chromium (III) benzoate and chromium (III) naphthenate. Of those, chromium (III)-2-ethylhexanoate is particularly preferred.

### (Nitrogen-Containing Compound (b))

The nitrogen-containing compound (b) used in the embodiment of the invention includes at least one compound selected from the group consisting of an amine, an amide and an imide. Examples of the amine include a primary amine compound, a secondary amine compound and a mixture of those. Examples of the amide include a metal amide compound derived from a primary amine compound or a secondary amide compound, a mixture of those, and an acid amide compound. Specific examples of the imide include 1,2-cyclohexanedicarboxylmide, succinimide, phthalimide, maleimide and those metal salts.

The preferred nitrogen-containing compound (b) used in the embodiment of the invention includes a secondary amine compound. Examples of the secondary amine compound include pyrroles such as pyrrole, 2,4-dimethylpyrrole, 2,5-dimethylpyrrole, 2-methyl-5-ethylpyrrole, 2,5-dimethyl-3-ethylpyrrole, 3,4-dimethylpyrrole, 3,4-dichloropyrrole, 2,3,4,5-tetrachloropyrrole and 2-acetylpyrrole, and their derivatives. Examples of the derivative include metal pyrrolide derivatives. Specific examples of the metal pyrrolide derivative include diethylaluminum pyrrolide, ethylaluminum dipyrrolide, aluminum tripyrrolide, sodium pyrrolide, lithium pyrrolide, potassium pyrrolide, diethylaluminum(2,5-dimethylpyrrolide), ethylaluminum bis(2,5-dimethylpyrrolide), aluminum tris(2,5-dimethyl-pyrrolide), sodium(2,5-dimethylpyrrolide), lithium(2,5-dimethylpyrrolide) and potassium(2,5-dimethylpyrrolide). Of those, 2,5-dimethylpyrrole and diethylaluminum(2,5-dimethylpyrrolide) are preferred. (Here, the aluminum pyrrolides are not included in the aluminum-containing compound (c). Furthermore, the halogen-containing pyrrole compound (b) is not included in the halogen-containing compound (d).)

### (Aluminum-Containing Compound (c))

The aluminum-containing compound (c) used in the embodiment of the invention includes at least one compound such as a trialkylaluminum compound, an alkoxyalkylaluminum compound and a hydrogenated alkylaluminum compound. Specific examples thereof include trimethylaluminum, triethylaluminum, triisobutylaluminum, diethylaluminum ethoxide and diethylaluminum hydride. Of those, triethylaluminum is particularly preferred.

### (Halogen-Containing Compound (d))

The chromium series catalyst used in the embodiment of the invention contains the halogen-containing compound (d) as a fourth component according to need. Examples of the halogen-containing compound (d) include at least one compound of a halogenated alkylaluminum compound, a linear halohydrocarbon with 2 or more carbon atoms having 3 or more halogen atoms and a cyclic halohydrocarbon with 3 or more carbon atoms having 3 or more halogen atoms. (The halogenated alkylaluminum compound is not included in the aluminum-containing compound (c)). Specific examples thereof include diethylaluminum chloride, ethylaluminum sesquichloride, carbon tetrachloride, 1,1,1-trichloroethane, 1,1,2,2-tetrachloroethane, pentachloroethane, hexachloro-ethane, 1,2,3-trichlorocyclopropane, 1,2,3,4,5,6-hexachlorocyclohexane and 1,4-bis(trichloromethyl)-2,3,5,6-tetrachlorobenzene.

In the embodiment of the invention, the low polymerization of an ethylene is preferably that the ethylene and the chromium series catalyst are contacted in an embodiment that the chromium compound (a) and the aluminum-containing compound (c) are not previously contacted, or the previous contact thereof is short. Such a contact embodiment makes it possible to selectively conduct trimerization reaction of ethylene, thereby obtaining 1-hexene from ethylene as a raw material in high yield.

The contact embodiment in the above continuous reaction system includes the following (1) to (9).

(1) A method of simultaneously introducing a mixture of the catalyst components (a), (b) and (d) and the catalyst component (c) into a reactor, respectively.

(2) A method of simultaneously introducing a mixture of the catalyst components (b) to (d) and the catalyst component (a) into a reactor, respectively.

(3) A method of simultaneously introducing a mixture of the catalyst components (a) and (b) and a mixture of the catalyst components (c) and (d) into a reactor, respectively.

(4) A method of simultaneously introducing a mixture of the catalyst components (a) and (d) and a mixture of the catalyst components (b) and (c) into a reactor, respectively.

(5) A method of simultaneously introducing a mixture of the catalyst components (a) and (b), the catalyst component (c) and the catalyst component (d) into a reactor, respectively.

(6) A method of simultaneously introducing a mixture of the catalyst components (c) and (d), the catalyst component (a) and the catalyst component (b) into a reactor, respectively.

(7) A method of simultaneously introducing a mixture of the catalyst components (a) and (d), the catalyst component (b) and the catalyst component (c) into a reactor, respectively.

(8) A method of simultaneously introducing a mixture of the catalyst components (b) and (c), the catalyst component (a) and the catalyst component (d) into a reactor, respectively.

(9) A method of simultaneously and independently introducing each of the catalyst components (a) to (d).

The above-described each catalyst component is generally dissolved in a solvent used in the reaction, and supplied to a reactor.

The “embodiment that the chromium compound (a) and the aluminum-containing compound (c) are not previously contacted” is not limited to the initiation time of the reaction, and means that such an embodiment is maintained even in the supply of the subsequent additional ethylene and catalyst component into the reactor.

Furthermore, in a batch reaction type, it is desired that the same embodiment is utilized.

The ratio of each constituent in the chromium series catalyst used in the embodiment of the invention is generally that the nitrogen-containing compound (b) is from 1 to 50 moles, and preferably from 1 to 30 moles, per mole of the chromium compound (a), and the aluminum-containing compound (c) is from 1 to 200 moles, and preferably from 10 to 150 moles, per mole of the chromium compound. Furthermore, the halogen-containing compound (d) is from 1 to 50 moles, and preferably from 1 to 30 moles, per mole of the chromium compound (a).

In the embodiment of the invention, the amount of the chromium series catalyst used is not particularly limited, but is generally from 1×10⁻⁷ to 0.5 mole, preferably from 5.0×10⁻⁷ to 0.2 mole, and further preferably from 1.00×10⁻⁶ to 0.05 mole, in terms of chromium atom of the chromium compound (a) per 1 liter of the solvent described hereinafter.

By using such a chromium series catalyst, hexene which is a trimer of ethylene can be obtained in selectivity of 90% or more. In this case, the proportion of 1-hexene occupied in hexene can be 99% or more.

### (Solvent)

In the production method of an ethylene low polymer to which the embodiment of the invention is applied, the low polymerization reaction of an ethylene can be conducted in a solvent.

Such a solvent is not particularly limited. However, for example, chain saturated hydrocarbons or alicyclic saturated hydrocarbons, having from 1 to 20 carbon atoms, such as butane, pentane, 3-methylpentane, hexane, heptane, 2-methylhexane, octane, cyclohexane, methylcyclohexane, 2,2,4-trimethylpentane and decalin; and aromatic hydrocarbons such as benzene, toluene, xylene, ethylbenzene, mesitylene and tetralin are used. Furthermore, an ethylene low polymer may be used as a solvent. Those can be used alone or as a mixed solvent.

In particular, the preferred solvent is chain saturated hydrocarbons or alicyclic saturated hydrocarbons, having from 4 to 10 carbon atoms. When those solvents are used, by-produced polymers such as a polyethylene can be suppressed. Furthermore, when the alicyclic saturated hydrocarbons are used, high catalyst activity tends to be obtained.

### (Production Method of Ethylene Low Polymer)

The ethylene low polymer in the present invention means a polymer comprising a plurality of ethylene as a monomer being bonded. Specifically, it means a polymer comprising 2 to 10 of ethylene as a monomer being bonded. The production method of an ethylene low polymer is described by referring to an example of the production of 1-hexene which is a trimer of ethylene as an ethylene low polymer.

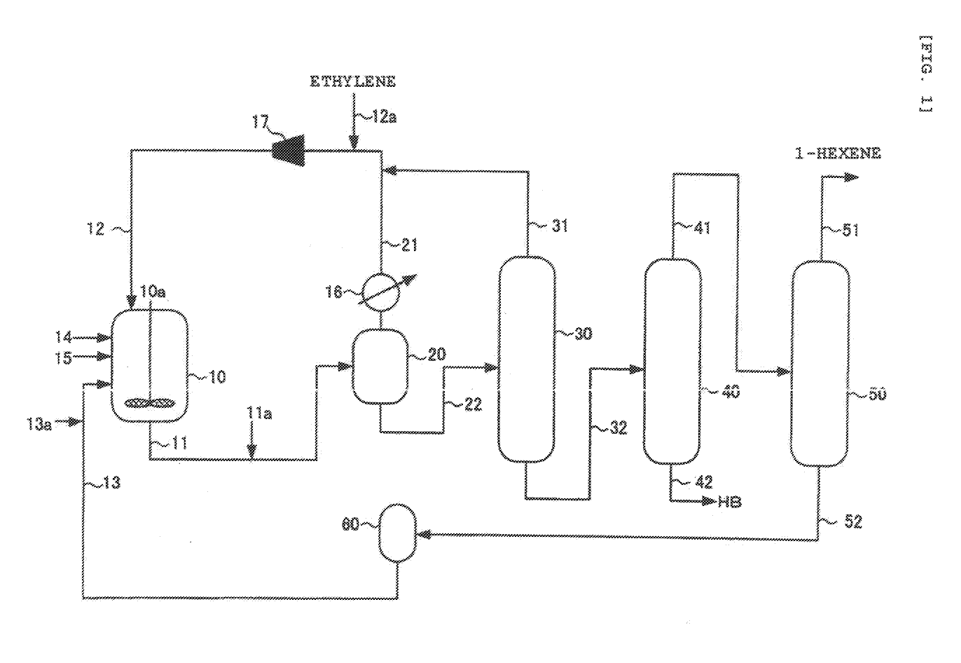

FIG. 1 is a view explaining a production flow example of an ethylene low polymer in the embodiment of the invention. The production flow example of 1-hexene using ethylene as a raw material shown in FIG. 1 shows a completely mixing and stirring type reactor 10 in which ethylene is subjected to low polymerization in the presence of a chromium series catalyst, a degassing tank 20 that separates an unreacted ethylene gas from a reaction liquid withdrawn from the reactor 10, an ethylene separation column 30 that distills ethylene in the reaction liquid withdrawn from the degassing tank 20, a high boiling separation column 40 that separates a solution containing decene, tetradecene and by-produced polymers (hereinafter this solution is referred to as “HB” (high boiler)) from the reaction liquid withdrawn from the ethylene separation column 30, and a hexene separation column 50 that distills the reaction liquid withdrawn from the top of the high boiling separation column 40 to distill away 1-hexene.

Furthermore, a compressor 17 that circulates an unreacted ethylene separated in the degassing tank 20 and the condenser 16 into the reactor 10 via a circulation piping 21 is provided.

The high boiler in the embodiment of the invention contains large amounts of decene, tetradecene and by-produced polymer that are by-products of low polymerization reaction of ethylene, and further contains a slight amount of catalyst components.

In FIG. 1, the reactor 10 includes the conventional reactors equipped with a stirring machine 10a, baffle, jacket and the like. As the stirring machine 10a, a stirring blade of the type such as paddle, pfaudler, propeller, turbine or the like is used in combination with a baffle such as a planar plate, a cylinder or a hairpin coil.

As shown in FIG. 1, ethylene is continuously supplied to the reactor 10 from an ethylene supply piping 12a via a compressor 17 and the first supply piping 12. Where the compressor 17 is, for example, a two-stage compression system, a circulation piping 31 is connected to the first stage, and a circulation piping 21 is connected to the second stage, thereby making it possible to reduce electric bill. On the other hand, the chromium compound (a) and the nitrogen-containing compound (b) are supplied from the second supply piping 13 via a catalyst supply piping 13a, the aluminum-containing compound (c) is supplied from the third supply piping 14, and the halogen-containing compound (d) is supplied from the fourth supply piping 15. Furthermore, a solvent used in low polymerization reaction of ethylene is supplied to the reactor 10 from the second supply piping 13.

In the embodiment of the invention, the reaction temperature in the reactor 10 is generally from 0 to 250° C., preferably from 50 to 200° C., and more preferably from 80 to 170° C.

The reaction pressure is in a range of generally from normal pressures to 250 kgf/cm², preferably from 5 to 150 kgf/cm², and more preferably from 10 to 100 kgf/cm².

The trimerization reaction of ethylene is preferably conducted such that a molar ratio of 1-hexene to ethylene in the reaction liquid ((1-hexene in reaction liquid)/(ethylene in reaction liquid)) is from 0.05 to 1.5, and particularly from 0.10 to 1.0. Specifically, it is preferred that in the case of a continuous reaction, a catalyst concentration, a reaction pressure and other conditions are adjusted such that the molar ratio of 1-hexene to ethylene in the reaction liquid is in the above range, and in the case of a batchwise reaction, the reaction is stopped at the time that the molar ratio is in the above range. This has the tendency that by-production of components having a boiling point higher than that of 1-hexene is suppressed, thereby further increasing selectivity of 1-hexene.

With respect to the reaction liquid continuously withdrawn from the bottom of the reactor 10 via a piping 11, trimerization reaction of ethylene is stopped by a deactivator supplied from a deactivator supply piping 11a, and such a reaction liquid is supplied to the degassing tank 20. In the degassing tank 20, unreacted ethylene is degassed from the top thereof, and circulated and supplied to the reactor 10 via the circulation piping 21, the condenser 16, the compressor 17 and the first supply piping 12. The reaction liquid from which unreacted ethylene has been degassed is withdrawn from the bottom of the degassing tank 20.

Operation conditions of the degassing tank 20 are that the temperature is generally from 0 to 250° C., and preferably from 50 to 200° C., and the pressure is generally from normal pressures to 150 kgf/cm², and preferably from normal pressures to 90 kgf/cm².

Subsequently, the reaction liquid from which unreacted ethylene gas has been degassed in the degassing tank 20 is withdrawn from the bottom of the degassing tank 20, and supplied to an ethylene separation column 30 by a piping 22. In the ethylene separation column 30, ethylene is distilled away from the column top by distillation, and circulated and supplied to the reactor 10 via the circulation piping 31 and the first supply piping 12. The reaction liquid from which ethylene has been removed is withdrawn from the bottom.

Operation conditions of the ethylene separation column 30 are that the top pressure is generally from normal pressures to 30 kgf/cm², and preferably from normal pressures to 20 kgf/cm², and the reflux ratio (R/D) is generally from 0 to 500, and preferably from 0.1 to 100.

The reaction liquid from which ethylene has been distilled in the ethylene separation column 30 is withdrawn from the bottom of the ethylene separation column 30, and supplied to a high boiling separation column 40 by a piping 32. In the high boiling separation column 40, a distillate containing 1-hexene which is an ethylene low polymer is withdrawn from the top by a piping 41. The high boiler is withdrawn from the bottom thereof and supplied to an evaporative separator (not shown) described hereinafter. Treatment of the high boiler in the evaporative separator is described hereinafter.

Operation conditions of the high boiling separation column 40 are that the top pressure is generally from 0.1 to 10 kgf/cm², and preferably from 0.5 to 5 kgf/cm², and the reflux ratio (R/D) is generally from 0 to 100, and preferably from 0.1 to 20.

Subsequently, the distillate containing 1-hexene withdrawn from the top of the high boiling separation column 40 is supplied to a hexene separation column 50 by the piping 41. In the hexene separation column 50, 1-hexene by distillation is distilled from the top by a piping 51. Heptane is withdrawn from the bottom of a hexene separation column 50, and stored in a solvent drum 60 via a solvent circulation piping 52, and circulated and supplied as a reaction solvent to the reactor 10 via the second supply piping 13.

Operation conditions of the hexene separation column 50 are that the top pressure is generally from 0.1 to 10 kgf/cm², and preferably from 0.5 to 5 kgf/cm², and the reflux ratio (R/D) is generally from 0 to 100, and preferably from 0.1 to 20.

### (Evaporative Separator)

Treatment of the high boiler withdrawn from the bottom of the high boiling separation column 40 is described below.

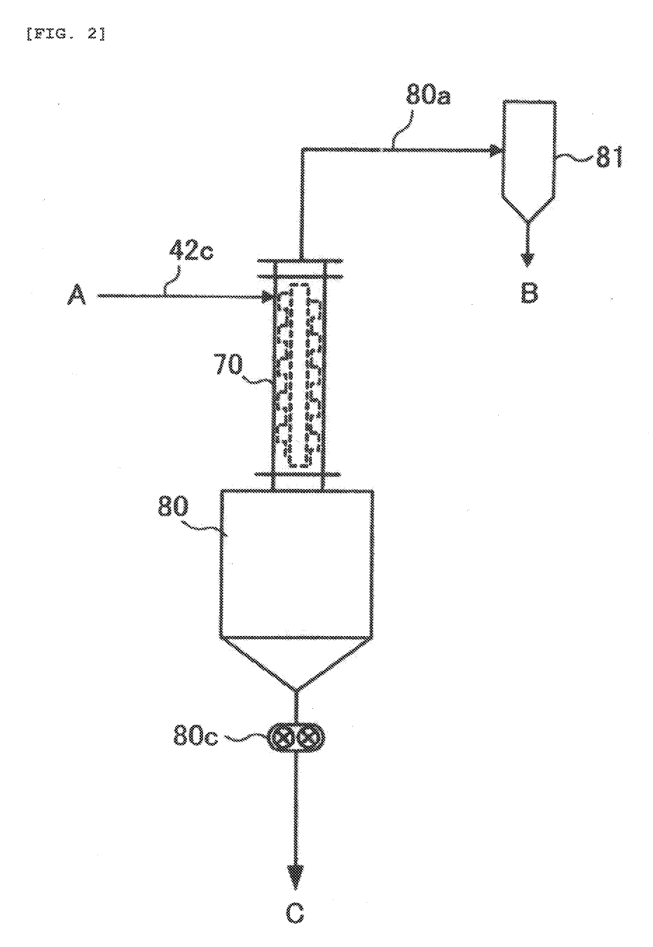

FIG. 2 is a view explaining a flow example of evaporative separation of a high boiler by an evaporative separator. The flow example of the evaporative separation shown in FIG. 2 shows an evaporative separator 70 which concentrates the high boiler withdrawn from the bottom of the high boiling separation column 40 (see FIG. 1) and additionally separates decene in the high boiler, a liquid storage tank 80 which stores a residual solution as a remainder of a high boiler of high viscosity containing concentrated tetradecene and by-produced polymer, and a gear pump 80c by which the residual solution is discharged.

Furthermore, as shown in FIG. 2, a condenser 81 which liquefies decene in a form of a gas evaporated and separated from the high boiler by the evaporative separator 70 is provided.

The evaporative separator 70 is not particularly limited, and can use the conventional various separators. For example, a packed column, a thin film evaporator equipped with a wiping blade which rotates relative to a heat transfer surface of a cylindrical inner mold, and the like; a wet wall evaporator; and the like are exemplified. In the embodiment of the invention, a thin film evaporator which can perform high concentration in further short period of time is used as the evaporative separator 70.

In FIG. 2, the high boiler from which components containing 1-hexene have been evaporated and separated, withdrawn from the bottom A of the high boiling separation column 40 (see FIG. 1) via a piping 42 is supplied to the evaporative separator 70, and decene in the high boiler is evaporated and separated by given operation conditions.

The high boiler supplied to the evaporative separator 70 is concentrated by that decene is evaporated and separated. The degree of concentration is such that the concentration of tetradecene contained in the residual solution is 5% by weight or more, and preferably 10% by weight or more. Where heat treatment is conducted under excessive high temperature conditions or in an excessively long period of time in order to increase the concentration rate (decene recovery rate), decomposition of catalyst components is accelerated, and the amount of introduction of a halogen compound and the like into decene evaporated and separated tends to be increased.

Examples of the chloro compound formed by heat decomposition of catalyst residues include 1-chloro-2-ethylhexyl, chlorodecane and chlorododecane.

Analysis was conducted with a gas chromatography having an atomic emission detector (chlorine atom). Chlorine concentration was calculated from the total area of peaks containing a chlorine atom.

In the embodiment of the invention, the high boiler from which an ethylene low polymer has been evaporated and separated evaporates and separates decene in the high boiler by the evaporative separator 70 so as to satisfy the following general expression (1).

By evaporating and separating decene from the high boiler by the evaporative separator 70 so as to satisfy the following general expression (1), the amount of a halogen contained in decene evaporated and separated is suppressed to a decomposition rate of 10% or less to the total amount of halogens contained in the high boiler supplied to the evaporative separator 70.

[Exp. 3]

θ/1.2EXP(800/T)≦1 (1)

(wherein T is temperature (° C.) of a residual solution and θ is residence time (min.) of a residual solution in an evaporative separator.)

The general expression (1) shows the relationship between the temperature and the residence time in the evaporative separator in condensing the high boiler to suppress formation of a chloro compound by heat decomposition of a chloro-containing catalyst residue. Reaction rate r of formation of a chloro compound formed by decomposition is defined r=kθ (k: rate constant, θ: residence time). The rate constant k has temperature dependency of formation reaction, and this dependency is expressed as 1.2EXP(800/T) of the term in the general expression (1) in terms of Arrhenius equation. Therefore, the decomposition rate expression of the chloro compound is represented by r=k′×1.2EXP(800/T)θ, and this means that where the temperature T and the residence time θ are determined, decomposition of the chloro compound can be suppressed.

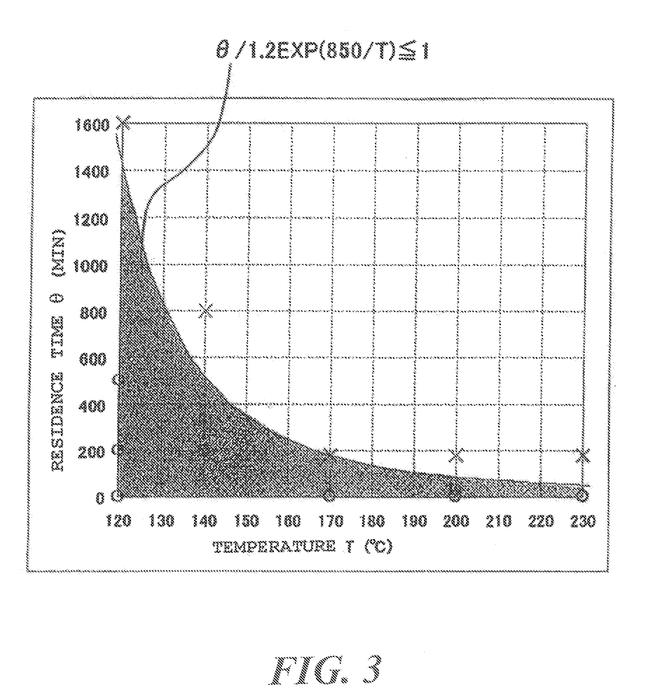

FIG. 3 is a view explaining the range of the general expression (1) that performs evaporative separation operation in the embodiment of the invention. As shown in FIG. 3, the range satisfying the general expression (1) that performs evaporative separation operation in the evaporative separator 70 and the liquid storage tank 80 is shown as a region shown by oblique lines in FIG. 3 when the temperature T (° C.) of the residual solution is a horizontal axis and the residence time θ (min.) of the residual solution in the evaporative separator 70 and the liquid storage tank 80 is a vertical axis.

In the embodiment of the invention, the temperature T for evaporating and separating decene in the high boiler in the evaporative separator 70 and the liquid storage tank 80 is generally from 80 to 230° C., and preferably from 100 to 200° C., on the assumption that the relationship of the above conditions is satisfied. Where the temperature for evaporation and separation is excessively high, decomposition of a chromium series catalyst and the like in the residual solution tends to be accelerated.

The residence time θ of the residual solution for evaporating and separating decene in the high boiler in the evaporative separator 70 is generally from 10 to 1,600 minutes, and preferably from 5 to 60 minutes, on the assumption that the relationship of the general expression (1) is satisfied. The residence time θ is excessively long, concentration of the residual solution proceeds, and heat transfer surface of the evaporative separator 70 tends to be fouled.

Gaseous decene evaporated and separated in the evaporative separator 70 is sent to a condenser 81 via a piping 80a. Liquid decene cooled in the condenser 81 is sent to B and recovered.

The residual solution of high viscosity stored in the liquid storage tank 80 is flown down from the bottom of the liquid storage tank 80 by plasticity of by-produced polymers contained, and discarded as an industrial waste C by a gear pump 80c.

### EXAMPLES

The present invention is described further specifically based on the Examples. However, the present invention is not limited to the following Examples so far as it does not depart from its gist.

### Reference Example 1

The following continuous low polymerization reaction of ethylene is carried out in a process having the reactor 10, the condenser 16, the degassing tank 20, the ethylene separation column 30, the high boiling column 40, the hexene separation column 50 and the solvent drum 60 which stores a circulation solvent, as shown in FIG. 1.

Regarding ethylene, unreacted ethylene separated from the degassing tank 20 and the ethylene separation column 30 is continuously supplied together with ethylene freshly supplied from the ethylene supply piping 12a to the reactor 10 from the first supply piping 12 by the compressor 17.

Regarding a solvent, a recovered n-heptane solvent separated in the hexene separation column 50 is passed through the solvent drum 60 (2 kgf/cm² nitrogen seal), and is continuously supplied to the reactor 10 from the second supply piping 13 at a flow rate of 40 liters/hr.

Next, regarding a catalyst, an n-heptane solution containing chromium (III) 2-ethylhexanoate (a) and 2,5-dimethylpyrrole (b) is continuously supplied from the catalyst supply piping 13a to the reactor 10 at a flow rate of 0.1 liter/hr via the second supply piping 13.

An n-heptane solution of triethylaluminum (c) is continuously supplied to the reactor 10 from the third supply piping 14 at a flow rate of 0.03 liter/hr. Furthermore, an n-heptane solution of hexachloroethane (d) is continuously supplied to the reactor 10 from the fourth supply piping 15 at a flow rate of 0.02 liter/hr.

The molar ratio of each component of the catalyst is (a):(b):(c):(d)=1:6:40:4. The solution of each component of the catalyst is supplied from a tank (not shown) sealed with nitrogen in 2 kgf/cm².

The reaction conditions of continuous low polymerization of ethylene in the reactor 10 are 120° C. and 51 kgf/cm².

2-Ethylhexanol as a metal solubilizing agent is added to the reaction liquid continuously withdrawn from the reactor 10 from the deactivator supply piping 11a at a flow rate of 0.005 liter/hr, and such a reaction liquid is then successively treated in the degassing tank 20, the ethylene separation column 30, the high boiling separation column 40 and the hexene separation column 50.

When the high boiler withdrawn from the bottom of the high boiling separation column 40 is analyzed with a gas chromatography (GC), decene is 95% by weight, and tetradecene is 2% by weight. The remainder of 3% by weight is other components such as by-produced polymers and catalyst components, that are not detected with GC.

### Examples 1 to 5, and Comparative Examples 1 to 5

### Preparation of High Boiler Raw Material

94 ml of 1.25 g-Cr/liter chromium series catalyst obtained by pre-adjusting chromium tris 2-ethylhexanoate (0.243 mmol), 2,5-dimethylpyrrole (1.46 mmol) and triethylaluminum (1.46 mmol) in a heptane solvent, 9.4 ml of a heptane solution of 100 g/liter triethylaluminum, 31.2 ml of a heptane solution of 7.37 g/liter hexachloroethane, and 6.8 ml of heptane were charged in a 300 ml SUS autoclave under nitrogen. The autoclave was sealed, the temperature of the autoclave was elevated to 140° C., and the autoclave was heated for 1 hours. Thereafter, 4.27 ml of 2-ethylhexanol was added to deactivate the catalyst, followed by further heating at 160° C. for 6 hours. Thereafter, the temperature was decreased to 95° C., and while flowing a nitrogen gas in the autoclave, heptane was distilled away, followed by drying until catalyst components are dried up to dryness. The solid thus obtained was used as a high boiler raw material.

### <Preparation of Sample>

2 ml of 1-decene and 0.2 g of the catalyst solid prepared above were charged in a 4 ml SUS small-sized vessel under nitrogen. Chlorine concentration in the 1-decene solution just after charging is zero.

### <Heat Treatment Test>

The vessel of the sample thus prepared was sealed, and the small-sized vessel was dipped in a sand bath adjusted to a given treatment temperature T (° C.), and heat treatment was conducted for the given treatment time θ (min.) while stirring the inner liquid by shaking the small-sized vessel up and down. Thereafter the small-sized vessel was dipped in a water bath to cool.

Analysis of chlorine concentration in the solution of the liquid after treatment was conducted under the following conditions using a gas chromatography equipped with an atomic emission detector (AED/GC).
Analyzer: Gas chromatography (Agilent 6890)
   Atomic emission detector (chlorine atom)
   Agilent G2350A (Cl 479 nm)
   Supelcowax-10, strong polarity, 0.32 mm, 60 m,
   0.25 μm
Measurement conditions: Gas He=40 cm/s
   Inlet temperature 250° C.
   Column temperature 50° C.→200° C.,
   10° C./min

Calibration for quantitatively determining chlorine concentration was conducted with a make-up liquid of trichloroethylene. In the sample analysis, chlorine concentration was calculated from the total area of peaks containing a chlorine atom. In this test, 1-chloro-2-ethylhexane, 3-chlorodecane and the like were detected as chlorine components. On the other hand, the chlorine concentration in the charged raw material was calculated from the charged amount of the chlorine-containing compound. The chlorine concentration in the case that the total amount of chlorine was present in a liquid after treatment was calculated as 285 ppm by weight. The decomposition rate (%) was calculated as chlorine concentration analysis value (wtppm) of sample/285 wtppm. This calculation was made to the samples prepared under the same conditions as above except for changing the treatment temperature T (° C.) and the treatment time θ (min.). The samples satisfying the expression (1) were as Examples 1 to 5, and the samples not satisfying the expression (1) were as Comparative Examples 1 to 5. The results are shown in Table 1. The “left side of general expression (1)” in Table 1 is the calculation result of the left side obtained by assigning the treatment temperature T (° C.) and the treatment time θ (min.) obtained in this test to the treatment temperature T (° C.) and the treatment time θ (min.) of the residual solution of the expression (1), respectively.

TABLE 1

| | | | | | | | Total chlorine |
|---|---|---|---|---|---|---|---|
| | | | | Total chlorine | | Left side | concentration |
| | | Treatment | Treatment | concentration | Decomposi- | in general | in high boiler |
| | | temperature | time θ | after treatment | tion rate | expression | raw material |
| | | T (° C.) | (min.) | (wtppm) | (%) | (1) | (wtppm) |
| Example | 1 | 120 | 200 | 11 | 3.9 | 0.14 | 285 |
| | | 120 | 500 | 11 | 3.9 | 0.35 | |
| | 2 | 140 | 200 | 20 | 7.0 | 0.38 | |
| | 3 | 170 | 5 | 12 | 4.2 | 0.03 | |
| | 4 | 200 | 5 | 5 | 1.8 | 0.06 | |
| | | 200 | 30 | 20 | 7.0 | 0.36 | |
| | 5 | 230 | 5 | 21 | 7.4 | 0.10 | |
| Compara- | 1 | 120 | 1600 | 30 | 10.5 | 1.12 | 285 |
| tive | 2 | 140 | 800 | 100 | 35.1 | 1.54 | |
| Example | 3 | 170 | 180 | 30 | 10.5 | 1.01 | |
| | 4 | 200 | 180 | 56 | 19.6 | 2.14 | |
| | 5 | 230 | 180 | 115 | 40.3 | 3.72 | |

It is seen from the results shown in Table 1 that when the samples are heat treated with the treatment temperature T (° C.) and the treatment time θ (min.) so as to satisfy the general expression (1), decomposition of chlorine contained in the high boiler raw material is less.

The above samples contain the catalyst components in an amount of about 10 times the amount of the catalyst component contained in the high boiler withdrawn from the bottom of the high boiling separation column 40 in Reference Example 1. It is apparent from the results shown in Table 1 that by evaporating and separating the high boiler of Reference Example 1 so as to satisfy the general expression (1) (Examples 1 to 5), the amount of chlorine contained in decene recovered is decreased, and the effect can be expected that the decomposition rate is suppressed to 10% or less to the amount of chlorine contained in the high boiler before evaporative separation operation.

On the other hand, it is seen that when evaporative separation operation is conducted without satisfying the general expression (1) (Comparative Examples 1 to 5), the amount of chlorine contained in decene recovered is increased.

While the invention has been described in detail and with reference to the specific embodiments thereof, it will be apparent to one skilled in the art that various changes and modifications can be made therein without departing from the spirit and scope thereof.

This application is based on Japanese Patent Application (Patent Application No. 2006-356464) filed Dec. 28, 2006, the entire contents thereof being hereby incorporated by reference.

### INDUSTRIAL APPLICABILITY

According to the present invention, decene having a small content of a decomposition product of a chromium series catalyst can be recovered from a residual solution (called a high boiler or a high boiling by-product liquid) from which an unreacted ethylene, 1-hexene and a solvent have been separated. Therefore, the industrial value of the present invention is remarkable.

1. A production method of an ethylene low polymer using a chromium series catalyst, comprising:
subjecting ethylene to low polymerization in a solvent in the presence of the chromium series catalyst,
separating an ethylene low polymer from a reaction liquid containing the ethylene low polymer to obtain a solution containing decene and tetradecene, and
separating and recovering decene from the solution containing decene and tetradecene by an evaporative separator under the condition of following general expression (1):
[Exp. 1]
θ/1.2EXP(850/T)≦1 (1)
wherein T is temperature (° C.) of a residual solution, and θ is residence time (min.) of a residual solution in an evaporative separator.

2. The production method of an ethylene low polymer as according to claim 1, wherein that the amount of a halogen contained in the decene separated from the evaporative separator is decomposition rate of 10% or less to the amount of a halogen contained in the residual solution.

3. The production method of an ethylene low polymer according to claim 1, wherein the chromium series catalyst is comprises a combination of at least (a) a chromium compound, (b) a nitrogen-containing compound, (c) an aluminum-containing compound and (d) a halogen-containing compound.

4. The production method of an ethylene low polymer according to claim 1, wherein the evaporative separator is a thin film evaporator.

5. The production method of an ethylene low polymer according to claim 1, wherein the ethylene low polymer is 1-hexene.
